(19) United States
(12) Patent Application Publication
(10) Pub. No.: US 2010/0137211 A1
(43) Pub. Date: Jun. 3, 2010

(54) METHODS AND COMPOSITIONS FOR INTRA-ARTICULAR COAGULATION PROTEINS

(76) Inventors: Paul E. Monahan, Chapel Hill, NC (US); Richard Jude Samulski, Chapel Hill, NC (US); Darrel W. Stafford, Carrboro, NC (US)

Correspondence Address: MYERS BIGEL SIBLEY & SAJOVEC, PO BOX 37428, RALEIGH, NC, 27627, US

(21) Appl. No.: 12/595,308

(22) PCT Filed: Apr. 11, 2008
(86) PCT No.: PCT/US08/04713

§ 371 Date: Oct. 9, 2009

Related U.S. Application Data
(60) Provisional application No. 60/922,780, filed on Apr. 11, 2007.

(51) Int. Cl. A61K 38/37; A61K 38/00; A61K 31/7088; A61K 38/36; A61K 38/19

(52) U.S. Cl. 514/12; 514/2; 514/44 R

(57) ABSTRACT

The present invention provides methods and compositions for treating blood clotting factor disorders and/or reducing bleeding-associated joint damage by treatments delivered to the joint in a subject.

### STATEMENT OF PRIORITY

This application claims the benefit, under 35 U.S.C. §119(e), of U.S. Provisional Application No. 60/922,780, filed Apr. 11, 2007, the entire contents of which are incorporated by reference herein.

### STATEMENT OF GOVERNMENT SUPPORT

Aspects of this invention were supported with funding provided under NIH NHLBI PPG: P01 HL66973. The U.S. Government has certain rights in this invention.

### BACKGROUND OF THE INVENTION

Severe blood clotting disorders result from the inherited or acquired deficiency of blood clotting factors. The most common severe deficiencies are those of coagulation factor VIII (hemophilia A) and factor IX (hemophilia B), although severe deficiencies of von Willebrand factor (Type 3 vWF) and factor XI (“hemophilia C”) may present with severe bleeding. Although life-threatening bleeding can occur, the major morbidity in these disorders comes from chronic recurrent bleeding into the joints. This joint bleeding results in a chronic inflammatory disorder known as hemophilic synovitis that in time leads to loss of function and eventual painful destruction of the joint, evolving into a complex arthritis known as hemophilic arthropathy (HA) in which the synovial disease is accompanied by degenerative changes in cartilage and underlying bone. As the inflammatory environment that develops in response to blood in a joint stimulates neoangiogenesis of fragile blood vessels, one or more “target” joints for recurrent bleeding develop. Greater than 80% of these joint bleeding episodes occur in six index joints: the knees, the elbows and the ankles. The natural history of joint disease in severe and moderate hemophilia is that, if not aggressively treated with large regular doses of intravenous clotting factor, progressive arthritis develops in one or more joints by the late teen years and deterioration is progressive. By age 25 years, 90% of persons with severe hemophilia (<1% factor VIII or factor IX activity) will have chronic degenerative changes in one to six joints.

Standard treatment consists of intravenous clotting factor infusions to attempt to raise the entire systemic circulating blood level of clotting factor, with the expectation that this strategy will also provide therapeutic clotting at the site of bleeding (e.g., the joint). Factor replacement in response to ongoing bleeding episodes does not halt the progression of existing arthropathy. Instead, institution of uninterrupted preventive (prophylactic) factor infusions, at an early age, prior to onset of recurrent joint bleeding, should be the standard of care. Mean annual costs for treating an adult with hemophilia are greater than $90,000/year, with costs typically doubling with development of a target joint and tripling if a full prophylactic treatment approach is used. Thus, the major costs of hemophilia to the health care system in dollars, to society in lost productivity, and to the person with hemophilia in terms of quality of life, result from bleeding into joints.

The assumption in the community of hematologists and researchers in the area of treatment of bleeding disorders has been that stable clot formation within the injured blood vessel is the only important site of action of the clotting factor. In addition, most coagulation physicians regard putting a needle into the joint of a hemophilic individual as something that should be avoided under most circumstances.

The present invention overcomes previous shortcomings in the art by providing methods and compositions for treatment of joint bleeding and bleeding disorders and reducing bleeding-associated joint damage.

### SUMMARY OF THE INVENTION

The present invention provides a method of treating a blood clotting factor disorder in a subject, comprising delivering an effective amount of a clotting factor protein and/or an active fragment thereof to a joint space of the subject (e.g., directly to the joint space), thereby treating the blood clotting factor disorder in the subject.

Further provided herein is a method of controlling bleeding and/or reducing bleeding-associated joint damage in a joint of a subject, comprising delivering an effective amount of a clotting factor protein and/or an active fragment thereof to a joint space of the subject (e.g., directly to the joint space), thereby controlling bleeding and/or protecting from further bleeding in the joint of the subject.

In addition, the present invention provides a method of treating a blood clotting factor disorder in a subject, comprising delivering an effective amount of a nucleic acid encoding a clotting factor protein and/or an active fragment thereof to a joint space of the subject (e.g., directly to the joint space), thereby treating the blood clotting factor disorder in the subject.

Furthermore, the present invention provides a method of controlling bleeding and/or reducing bleeding-associated joint damage in a joint of a subject, comprising delivering an effective amount of a nucleic acid encoding a clotting factor protein or an active fragment thereof to a joint space of the subject (e.g., directly to the joint space), thereby controlling bleeding in the joint space of the subject.

In additional embodiments, the present invention provides a method of treating or preventing hemophilic arthropathy or bleeding-associated joint damage in a subject, comprising delivering an effective amount of a clotting factor protein and/or an active fragment thereof to a joint space of the subject (e.g., directly to the joint space), thereby treating or reducing hemophilic arthropathy or bleeding-associated joint damage in the subject.

Also provided herein is a method of treating or preventing hemophilic arthropathy or bleeding-associated joint damage in a subject, comprising delivering an effective amount of a nucleic acid encoding a clotting factor protein and/or an active fragment thereof to a joint space of the subject (e.g., directly to the joint space), thereby treating or reducing hemophilic arthropathy or bleeding-associated joint damage in the subject.

In various embodiments, the methods of this invention can further comprise delivering an effective amount of a nucleic acid encoding a clotting factor protein or an active fragment thereof to a joint space of the subject. In some embodiments, the methods of this invention can further comprise systemically delivering an effective amount of a clotting factor protein and/or an active fragment thereof to the subject and in some embodiments, the methods of this invention can further comprise systemically delivering an effective amount of a nucleic acid encoding a clotting factor protein and/or an active fragment thereof to the subject. In further embodiments, the methods of this invention can further comprise delivering an effective amount of an anti-inflammatory agent, a cytokine, an immune modulator or any combination thereof to the subject. The methods of this invention can also further comprise delivering an effective amount of a nucleic acid encoding an anti-inflammatory agent, a cytokine, an immune modulator or any combination thereof to the subject.

Additional aspects of this invention include a composition comprising a clotting factor protein and/or active fragment thereof and a nucleic acid encoding a clotting factor protein and/or active fragment thereof, in a pharmaceutically acceptable carrier. A composition of this invention can further comprise an anti-inflammatory agent, a cytokine, an immune modulator, or any combination thereof and/or a nucleic acid encoding an anti-inflammatory agent, a cytokine, an immune modulator, or any combination thereof.

Additionally provided herein is a composition comprising a clotting factor protein and/or active fragment thereof and an anti-inflammatory agent, a cytokine, an immune modulator, or any combination thereof, in a pharmaceutically acceptable carrier.

Further provided herein is a composition comprising a nucleic acid encoding a clotting factor protein and/or active fragment thereof and a nucleic acid encoding an anti-inflammatory agent, a cytokine, an immune modulator, or any combination thereof, in a pharmaceutically acceptable carrier.

### BRIEF DESCRIPTION OF THE DRAWINGS

FIG. 1 shows that intra-articular delivery of FIX affords protection from synovitis when compared with systemic administration of FIX. FIX−/−mice received either IV recombinant hFIX followed by needle puncture and normal saline (NS) or needle puncture with coincident IA injection of hFIX. Fourteen days after injury, both left and right knee joints were collected for histological examination using a validated mouse hemophilic synovitis grading system. A grade of zero to ten is awarded for increasing pathology based on parameters of synovial hyperplasia (0-3 points), vascularity (0-3 points), or the presence of blood, synovial villus formation, discoloration by hemosiderin, or cartilage erosion ( )absent; 1=present). Average scores with the standard error of the mean are shown. N≧3 animals in each group. Differences between IA treatments of 20 IU/kg and 10 IU/kg, when compared to 100 IU/kg IV, were statistically significant (P<0.001 and <0.05, respectively).

FIG. 2 shows a quantitative analysis of cells in cartilage and synovium expressing hFIX following AAV2-, AAV5-, or AAV8-CBA-hFIX transduction. Data are presented as percentage of hFIX positive staining cells.

FIG. 3 shows the histopathologic grading of mouse joints treated with AAV.hFIX intra-articular delivery and comparison with pathology score of contralateral injured untreated knee. High dose: 1.0×10¹⁰ particles/animal; low dose: 2.5×10⁹ particles/animal. *P<0.05.

### DETAILED DESCRIPTION OF THE INVENTION

The present invention is based, in part, on the unexpected discovery that the delivery of clotting factor proteins into a joint space can treat and/or control bleeding into a joint space in a subject, such as a subject with a bleeding disorder. The methods of this invention can also be employed to prevent or reduce the incidence of bleeding episodes and/or the severity and long term consequences (e.g., hemophilic arthropathy) of such bleeding episodes in a subject of this invention, as well as to reduce the risk of future bleeding episodes in high-risk situations, such as peri-operatively. In some embodiments, the clotting factor proteins and/or nucleic acids of this invention can be delivered directly (e.g., via injection) to the joint space and/or joint tissue of the subject. In other embodiments, nucleic acid encoding coagulation protein(s) or clotting factor protein(s) can be delivered to a subject (e.g., at the joint and/or systemically), the subsequent expression of which results in the production of coagulation protein(s) or clotting factor protein(s) that act at the joint to prevent and/or control bleeding and/or bleeding-related joint damage.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and, variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, nucleotide sequences, amino acid sequences and other references mentioned herein are incorporated by reference in their entirety.

The designation of all amino acid positions in the AAV capsid subunits in the description of the invention and the appended claims is with respect to VP1 capsid subunit numbering.

Except as otherwise indicated, standard methods known to those skilled in the art may be used for the construction of rAAV constructs, modified capsid proteins, packaging vectors expressing the parvovirus rep and/or cap sequences, and transiently and stably transfected packaging cells. Such techniques are known to those skilled in the art. See, e.g., SAMBROOK et al., MOLECULAR CLONING: A

LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

### Definitions

The following terms are used in the description herein and the appended claims:

As used herein, “a,” “an,” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, “nucleic acid,” “nucleotide sequence” and “polynucleotide” encompass both RNA and DNA, including cDNA, genomic DNA, mRNA, synthetic (e.g., chemically synthesized) DNA and chimeras of RNA and DNA. The term polynucleotide or nucleotide sequence refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid or nucleotide sequence of this invention.

An “isolated nucleic acid” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found as such in the natural state. “Isolated” does not mean that the preparation is technically pure (homogeneous), but it is sufficiently pure to provide the polypeptide or nucleic acid in a form in which it can be used for the intended purpose.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated ex vivo and then returned to the subject.

The term “nucleic acid fragment” will be understood to mean a nucleotide sequence of reduced length relative to a reference nucleic acid or nucleotide sequence and comprising, consisting essentially of and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially or and/or consist of, oligonucleotides having a length of at least about 6, 7, 8, 9, 10, 11, 12, 13, 14, 15, 16, 17, 18, 19, 20, 21, 22, 23, 24, 25. 50, 75, 100, 150, 200, 250, 300, 350, 400, 450, 500, 750, 1000, 1500, 2000, 2500, 3000, 4000 or 5000 consecutive nucleotides of a nucleic acid or nucleotide sequence according to the invention.

Several methods known in the art may be used to produce a polynucleotide and/or vector according to this invention. A “vector” is any nucleic acid molecule for the cloning and/or amplification of nucleic acid as well as for the transfer of nucleic acid into a subject (e.g., a cell of the subject). A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral nucleic acid molecules for introducing a nucleic acid into a cell in vitro, ex vivo, and/or in vivo.

A large number of vectors known in the art may be used to manipulate nucleic acids, incorporate response elements and promoters into genes, etc. Such vectors include, for example, plasmids or modified viruses including, for example bacteriophages such as lambda derivatives, or plasmids such as pBR322 or pUC plasmid derivatives, or the Bluescript® vector. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker.

Vectors have been used in a wide variety of gene delivery applications in cells, as well as in living animal subjects. Viral vectors that can be used include but are not limited to retrovirus, lentivirus, adeno-associated virus, poxvirus, alphavirus, baculovirus, vaccinia virus, herpes virus, Epstein-Barr virus, and adenovirus vectors, as well as any combination thereof. Nonlimiting examples of non-viral vectors include plasmids, liposomes, electrically charged lipids (cytofectins), nucleic acid-protein complexes, and biopolymers, as well as any combination thereof. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, and/or a nucleic acid vector transporter (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990).

In some embodiments, a polynucleotide of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Feigner et al., Proc. Natl. Acad. Sci. USA 84:7413 (1987); Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Feigner et al., Science 337:387 (1989)). Particularly useful lipid compounds and compositions for transfer of nucleic acids are described in International Patent Publications WO 95/18863 and WO 96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules could be coupled to liposomes chemically.

In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931).

It is also possible to deliver a nucleotide to a subject in vivo as naked nucleic acid (see U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J. Biol. Chem. 262:4429 (1987)).

The term “transfection” means the uptake of exogenous or heterologous nucleic acid (RNA and/or DNA) by a cell. A cell has been “transfected” with an exogenous or heterologous nucleic acid when such nucleic acid has been introduced or delivered inside the cell. A cell has been “transformed” by exogenous or heterologous nucleic acid when the transfected nucleic acid imparts a phenotypic change in the cell and/or in an activity or function of the cell. The transforming nucleic acid can be integrated (covalently linked) into chromosomal DNA making up the genome of the cell and/or it can be present as a stable plasmid.

As used herein, “transduction” of a cell means the transfer of genetic material into the cell by the incorporation of nucleic acid into a virus particle and subsequent transfer into the cell via infection of the cell by the virus particle.

As used herein, the term “polypeptide” encompasses both peptides and proteins, unless indicated otherwise.

A “polynucleotide” or “nucleotide sequence” is a sequence of nucleotide bases, and may be RNA, DNA or DNA-RNA hybrid sequences (including both naturally occurring and non-naturally occurring nucleotides), but are typically either single or double stranded DNA sequences.

A “therapeutic polypeptide” is a polypeptide that can alleviate or reduce symptoms that result from an absence or defect in a protein in a cell or subject.

Alternatively, a “therapeutic polypeptide” is a polypeptide that otherwise confers a benefit to a subject, e.g., anti-cancer effects or improvement in transplant survivability.

The term “therapeutically effective amount” or “effective amount,” as used herein, refers to that amount of a composition of this invention that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, a therapeutically effective amount or effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

“Treat” or “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art.

By the terms “treat,” “treating” or “treatment of” (or grammatically equivalent terms) it is also meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder. In certain embodiments, the methods of this invention can be employed to prevent or minimize bleeding into joints and/or to prevent and/or minimize the severity and/or long term consequence of joint bleeding.

By “prevent,” “preventing” or “prevention” is meant to avoid or eliminate the development and/or manifestation of a pathological state and/or disease condition or status in a subject. For example, in the present invention, the prevention of bleeding associated joint damage means the avoidance or elimination of a bleeding event or episode that would result in bleeding associated joint damage.

In particular embodiments, the present invention provides a composition comprising, consisting essentially of and/or consisting of a protein and/or nucleic acid of this invention in a pharmaceutically acceptable carrier and, optionally, further comprising other medicinal agents, pharmaceutical agents, stabilizing agents, buffers, carriers, adjuvants, diluents, etc. In some embodiments, a composition of this invention can comprise, consist essentially of and/or consist of a protein and/or nucleic acid and/or vector of this invention in combination with an anti-inflammatory agent, a cytokine, an immune modulator, a locally acting analgesic (e.g., lidocaine), a coagulation-regulated anti-inflammatory agent (e.g., protease activator receptor 1, or thrombin receptor) of this invention. In some embodiments, a composition of this invention can comprise, consist essentially of and/or consist of a protein and/or nucleic acid and/or vector of this invention in combination with a nucleic acid encoding an anti-inflammatory agent and/or cytokine of this invention.

For injection, the carrier will typically be a liquid. For other methods of administration, the carrier may be either solid or liquid. For inhalation administration, the carrier will be respirable, and will preferably be in solid or liquid particulate form. Further provided herein is a pharmaceutical composition comprising a protein or active fragment thereof of this invention in a pharmaceutically acceptable carrier. Additional compositions of this invention can include any of the proteins, active fragments and/or nucleic acids of this invention in any combination, in a pharmaceutically acceptable carrier.

“Pharmaceutically acceptable,” as used herein, means a material that is not biologically or otherwise undesirable, i.e., the material may be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; latest edition). Exemplary pharmaceutically acceptable carriers for the compositions of this invention include, but are not limited to, sterile pyrogen-free water and sterile pyrogen-free physiological saline solution.

A further aspect of the invention is a method of administering or delivering a protein and/or nucleic acid of the invention to subjects. Administration or delivery to a human subject or an animal in need thereof can be by any means known in the art for administering proteins and/or nucleic acids. In some embodiments, the protein and/or nucleic acid is delivered in a therapeutically effective dose in a pharmaceutically acceptable carrier.

Dosages of virus particles to be administered to a subject will depend upon the mode of administration, the disease or condition to be treated, the individual subject's condition, the particular virus vector, and the nucleic acid to be delivered, and can be determined in a routine manner. Exemplary doses are virus titers of at least about 10⁵, 10⁶, 10⁷, 10⁸, 10⁹, 10¹⁰, 10¹¹, 10¹², 10³, 10¹⁴, 10¹⁵ transducing units or more, preferably about 10⁸-10¹³transducing units, yet more preferably 10¹² transducing units.

In particular embodiments, more than one administration (e.g., two, three, four or more administrations) of the nucleic acid or vector may be employed to achieve the desired level of gene expression over a period of various intervals, e.g., daily, weekly, monthly, yearly, etc.

Exemplary modes of administration of the proteins, nucleic acids and vectors of this invention can include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration, and the like, as well as direct tissue or organ injection (e.g., to liver, skeletal muscle, cardiac muscle, diaphragm muscle or brain). Administration can also be to a tumor (e.g., in or a near a tumor or a lymph node). The most suitable route in any given case will depend on the nature and severity of the condition being treated and on the nature of the particular protein, nucleic acid or vector that is being used.

As described in the embodiments herein, a clotting factor protein or coagulation protein or active fragment thereof can be administered directly into the joint space of a subject according to the methods of this invention as described herein. In certain embodiments, the clotting factor protein or coagulation protein or active fragment thereof will be present in a pharmaceutical composition further comprising a pharmaceutically acceptable carrier. Dosages of the clotting factor protein or active fragment thereof to be administered to a subject will depend upon the mode of administration, the disease or condition to be treated, the individual subject's condition, the particular clotting factor protein or coagulation protein, and any other agents being administered to the subject and can be determined in a routine manner according to methods well known in the art. An exemplary dosage range is from about 5 Units/kilogram of body weight (U/kg) to about 200 U/kg or from about 30 micrograms/kilogram of body weight to about 270 micrograms/kg or a dose calculated, according to art-known methods, to achieve a plasma or synovial fluid level of about 0.01 Unit/ml to 1.0 Unit/ml, depending on the protein being delivered.

In particular embodiments, more than one administration (e.g., two, three, four or more administrations) of the protein or active fragment thereof may be employed to achieve the desired result over a period of various intervals, e.g., daily, weekly, monthly, yearly, etc.

“Promoter” refers to a nucleic acid sequence capable of controlling the expression of a coding sequence or functional RNA. In general, a coding sequence is located 3′ to a promoter sequence. Promoters may be derived in their entirety from a native sequence, or be composed of different elements derived from different promoters found in nature, or even comprise synthetic nucleic acid segments. It is understood by those skilled in the art that different promoters may direct the expression of a nucleotide sequence in different tissues or cell types, or at different stages of development, or in response to different environmental or physiological conditions. Promoters that cause a nucleotide sequence to be expressed in most cell types at most times are commonly referred to as “constitutive promoters.” Promoters that cause a nucleotide sequence to be expressed in a specific cell type are commonly referred to as “cell-specific promoters” or “tissue-specific promoters.” Promoters that cause a nucleotide sequence to be expressed at a specific stage of development or cell differentiation are commonly referred to as “developmentally-specific promoters” or “cell differentiation-specific promoters.” Promoters that are induced and cause a nucleotide sequence to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, nucleotide sequences of different lengths may have identical promoter activity.

A “promoter sequence” is a nucleic acid regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence can be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” are nucleic acid regulatory sequences, such as promoters, enhancers, terminators, and the like, that provide for the expression of a coding sequence in a cell. For example, in eukaryotic cells, polyadenylation signals are control sequences.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., the coding sequence is under the transcriptional control of the promoter). Coding sequences can be operably linked to regulatory sequences in sense and/or antisense orientation.

The nucleic acids or plasmids or vectors may further comprise at least one promoter suitable for driving expression of a nucleotide sequence in a cell. The term “expression vector” means a vector, plasmid or vehicle designed to enable the expression of an inserted nucleotide sequence following delivery of a nucleotide sequence into a cell. The cloned nucleotide sequence, i.e., the inserted nucleotide sequence, is usually placed under the control of control elements such as a promoter, a minimal promoter, an enhancer, or the like. Initiation control regions or promoters, which are useful to drive expression of a nucleic acid in a cell are numerous and familiar to those skilled in the art. Virtually any promoter capable of driving expression of a nucleotide sequence is suitable for the present invention, including but not limited to: viral promoters, bacterial promoters, animal promoters, mammalian promoters, synthetic promoters, constitutive promoters, tissue specific promoters, developmental specific promoters, inducible promoters, and/or light regulated promoters.

The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, and B19 virus. Other autonomous parvoviruses are known to those skilled in the art. See, e.g., BERNARD

N. FIELDS et al., VIROLOGY, volume 2, chapter 69 (4th ed., Lippincott-Raven Publishers).

The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV or any other AAV now known or later discovered. See, e.g., BERNARD N. FIELDS et al., VIROLOGY, volume 2, chapter 69 (4th ed., Lippincott-Raven Publishers). Recently, a number of new AAV serotypes and clades have been identified (see, e.g., Gao et al., (2004) J. Virology 78:6381-6388 and Table 3).

The genomic sequences of the various serotypes of AAV and the autonomous parvoviruses, as well as the sequences of the terminal repeats (TRs), Rep proteins, and capsid subunits are known in the art. Such sequences may be found in the literature or in public databases such as GenBank®. See, e.g., GenBank Accession Numbers NC 002077, NC 001401, NC 001729, NC 001863, NC 001829, NC 001862, NC 000883, NC 001701, NC 001510, AF063497, U89790, AF043303, AF028705, AF028704, J02275, J01901, J02275, X01457, AF288061, AH009962, AY028226, AY028223, NC 001358, NC 001540, AF513851, AF513852, AY530579, AY631965, AY631966; the disclosures of which are incorporated by reference herein in their entirety. See also, e.g., Srivistava et al., (1983) J. Virology 45:555; Chiorini et al., (1998) J. Virology 71:6823; Chiorini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Virol. 58:921; Gao et al., (2002) Proc. Nat. Acad. Sci. USA 99:11854; international patent publications WO 00/28061, WO 99/61601, WO 98/11244; U.S. Pat. No. 6,156,303; the disclosures of which are incorporated by reference herein in their entirety. See also Table 3. An early description of the AAV1, AAV2 and AAV3 terminal repeat sequences is provided by Xiao (1996), “Characterization of adeno-associated virus (AAV) DNA replication and integration,” Ph.D. Dissertation, University of Pittsburgh, Pittsburgh, Pa. (incorporated herein by reference in its entirety).

The term “tropism” as used herein refers to preferential entry of the virus into certain cell or tissue types or preferential interaction with the cell surface that facilitates entry into certain cell or tissue types, optionally and preferably followed by expression (e.g., transcription and, optionally, translation) of sequences carried by the viral genome in the cell, e.g., for a recombinant virus, expression of the heterologous nucleotide sequence(s). Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of a rAAV genome, gene expression from the viral genome may be from a stably integrated provirus, from a non-integrated episome, as well as any other form that the virus nucleic acid may take within the cell.

A “heterologous nucleotide sequence” or “heterologous nucleic acid” is typically a sequence that is not naturally occurring in the virus genome in which it is present and/or is not naturally occurring in the cell into which it is introduced or is not naturally occurring in the cell into which it is introduced in the form and/or amount in which it is present in the cell. Generally, the heterologous nucleic acid or nucleotide sequence comprises an open reading frame that encodes a peptide, a polypeptide and/or a nontranslated functional RNA.

In certain embodiments described herein, the term “vector” or “delivery vector” can refer to a parvovirus (e.g., AAV) particle that functions as a nucleic acid delivery vehicle, and which comprises viral DNA (i.e., the vector genome) packaged within a parvovirus (e.g., AAV) capsid. In some contexts, the term “vector” may be used to refer to the vector genome/vDNA in the absence of the capsid. In some embodiments, the viral genome can be present in a different virus vector or in a non-viral vector.

As used herein, a “recombinant parvovirus vector genome” is a parvovirus genome (i.e., vDNA) that comprises at least one terminal repeat (e.g., two terminal repeats) and one or more heterologous nucleotide sequences. A “recombinant parvovirus particle” comprises a recombinant parvovirus vector genome packaged within a parvovirus capsid.

A “rAAV vector genome” or “rAAV genome” is an AAV genome (i.e., vDNA) that comprises at least one terminal repeat (e.g., two terminal repeats) and one or more heterologous nucleotide sequences. rAAV vectors generally require only the 145 base terminal repeat(s) (TR(s)) in cis to generate virus. All other viral sequences are dispensable and may be supplied in trans (Muzyczka, (1992) Curr. Topics Microbiol. lmmunol. 158:97). Typically, the rAAV vector genome will only retain the minimal TR sequence(s) so as to maximize the size of the transgene that can be efficiently packaged by the vector. The structural and non-structural protein coding sequences may be provided in trans (e.g., from a vector, such as a plasmid, or by stably integrating the sequences into a packaging cell). The rAAV vector genome optionally comprises two AAV TRs, which generally will be at the 5′ and 3′ ends of the heterologous nucleotide sequence(s), but need not be contiguous thereto. The TRs can be the same or different from each other.

A “rAAV particle” comprises a rAAV vector genome packaged within an AAV capsid.

A “parvovirus terminal repeat” may be from any parvovirus, including autonomous parvoviruses and AAV (all as defined above). An “AAV terminal repeat” may be from any AAV, e.g., serotypes 1, 2, 3, 4, 5, 6, 7, 8, 9, 10 and 11. The term “terminal repeat” includes synthetic sequences that function as an AAV inverted terminal repeat, such as the “double-D sequence” as described in U.S. Pat. No. 5,478,745 to Samulski et al., the disclosure of which is incorporated in its entirety herein by reference. The AAV terminal repeats need not have a wild-type terminal repeat sequence (e.g., a wild-type sequence may be altered by insertion, deletion, truncation or missense mutations), as long as the terminal repeat mediates the desired functions, e.g., replication, virus packaging, integration, and/or provirus rescue, and the like. In some embodiments, synthetic sequences or non-parvovirus TRs (e.g., SV40) can be used.

The capsid structure of autonomous parvoviruses and AAV is described in more detail in BERNARD N. FIELDS et al., VIROLOGY, volume 2, chapters 69 & 70 (4th ed., Lippincott-Raven Publishers).

The virus vector of the invention can further be a “targeted” virus vector (e.g., having a directed tropism) and/or a “hybrid” parvovirus (i.e., in which the rAAV genome and viral capsid are from different parvoviruses) as described in international patent publication WO 00/28004 and Chao et al. (2000) Molecular Therapy 2:619. In particular embodiments, the rAAV genome and virus capsid are from different AAV.

In particular embodiments, all of the subunits of the virus capsid are derived from the same AAV capsid protein backbone. In other embodiments, the virus capsid comprises capsid proteins that are derived from different AAV backbones.

The virus vectors of the invention can further be duplexed parvovirus particles comprising a non-resolvable terminal repeat, e.g., as described in International Patent Publication No. WO 01/92551.

Accordingly, as used herein, the terms “chimeric parvovirus” and “chimeric AAV” encompass hybrid, targeted and duplexed virus particles, as well as other modified forms of parvoviruses and AAV.

### Embodiments of Methods of the Invention

In one aspect, the present invention provides a method of treating a blood clotting factor disorder in a subject, comprising delivering an effective amount of a clotting factor protein or an active fragment thereof directly to a joint of the subject, thereby treating the blood clotting factor disorder in the subject. A clotting factor disorder is an abnormality of the body's normal balance of hemostasis and thrombosis, resulting from an abnormal body level of any of a number of procoagulant or anticoagulant proteins (or regulators of the activity of procoagulant or anticoagulant proteins), fibrinolytic proteins, or coagulation protein-regulated proteins; a clotting factor disorder results in an increased risk of either abnormal or abnormally increased bleeding or abnormal or abnormally increased pathologic thrombus formation. Classic examples of clotting factor disorders are hemophilia A and hemophilia B, which result from deficient activity of blood procoagulant proteins factor VIII and IX, respectively.

Further provided herein is a method of controlling bleeding in a joint space of a subject, comprising delivering an effective amount of a clotting factor protein or an active fragment thereof directly to the joint space of the subject, thereby controlling bleeding in the joint space of the subject. A method is also provided herein, of treating a blood clotting factor disorder in a subject, comprising delivering an effective amount of a nucleic acid encoding a clotting factor protein or an active fragment thereof directly to a joint space of the subject, thereby treating the blood clotting factor disorder in the subject.

In additional embodiments, the present invention provides a method of controlling bleeding in a joint space of a subject, comprising delivering an effective amount of a nucleic acid encoding a clotting factor protein or an active fragment thereof directly to the joint space of the subject, thereby controlling bleeding in the joint space of the subject.

Further aspects of this invention include a method of reducing bleeding in a subject, comprising delivering an effective amount of a nucleic acid encoding a clotting factor protein to the joint space of the subject, thereby reducing bleeding both within and at anatomic sites distant from the joint space of the subject (e.g., delivery of an AAV8 vector to the joint can result in effective transduction of cells both at the site of injection in the joint as well as in the liver, following vector spread out of the site of the injection to transduce cells in the liver).

Additionally provided herein is a method of treating or preventing hemophilic arthropathy in a subject, comprising delivering an effective amount of a clotting factor protein or an active fragment thereof to a joint space of the subject, thereby treating or preventing hemophilic arthropathy in the subject.

The present invention also provides a method of treating or preventing hemophilic arthropathy in a subject, comprising delivering an effective amount of a nucleic acid encoding a clotting factor protein or an active fragment thereof to a joint space of the subject, thereby treating or preventing hemophilic arthropathy in the subject.

Further provided herein is a method of treating or preventing hemophilic arthropathy in a subject, comprising delivering an effective amount of a nucleic acid encoding a clotting factor protein or an active fragment thereof to the subject, thereby treating or preventing hemophilic arthropathy in the subject.

“Coagulation proteins” or “clotting factor proteins” are soluble and tissue-bound proteins that maintain the body's normal balance of hemostasis and thrombosis and include, e.g., procoagulant and anticoagulant proteins (and regulators of the activity of procoagulant or anticoagulant proteins), fibrinolytic proteins, and coagulation protein-regulated proteins. In the methods and compositions of this invention, the coagulation protein or clotting factor protein can be, but is not limited to, Factor VII, Factor VIIA (activated), Factor VIII, Factor IX, Factor IX (activated), Factor X, Factor X (activated), Factor XI, von Willebrand factor, Protein C, activated Protein C, Protein S, bone Gla protein (osteocalcin), matrix Gla protein, prothrombin, thrombin, tissue factor pathway inhibitor (TFPI), antagonist of tissue factor pathway inhibitor, tissue factor, thrombin-associated fibrinolysis inhibitor (TAFI), antagonist of thrombin-associated fibrinolysis inhibitor (TAFI), protease-activated thrombin receptor (PAR-1), inhibitor of protease-activated thrombin receptor (PAR-1), protease-activated receptor 2 (PAR-2), inhibitor of protease-activated receptor 2 (PAR-2), protease-activated receptor 4 (PAR-4), inhibitor of protease-activated receptor 4 (PAR-4), other fibrinolytic proteins (e.g. epsilon-aminocaproic acid, tranexamic acid) and any combination thereof.

These clotting factor or coagulation proteins are well known in the art and the coding sequences of these proteins, as well as allelic or polymorphic variants of these proteins and the nucleotide sequences encoding them are readily available to one of ordinary skill in the art through such resources as online sequence databases (e.g., the GenBank® database). All such sequences are incorporated by reference herein in their entireties and are all intended to be within the scope of this invention. In the case of polypeptide sequences that are less than 100% identical to a reference sequence, the non-identical positions are preferably, but not necessarily, conservative substitutions for the reference sequence. Conservative substitutions are well known in the art and typically include, but are not limited to, substitutions within the following groups: glycine and alanine; valine, isoleucine, and leucine; aspartic acid and glutamic acid; asparagine and glutamine; serine and threonine; lysine and arginine; and phenylalanine and tyrosine.

The invention further provides additional variants and/or mutants of the clotting factor proteins or coagulation proteins that have modifications in the amino acid sequence that result in greater activity, reduced activity, decreased thrombotic risk, greater tissue longevity and/or improved tissue localization or tropism, in any combination, as compared with wild type or non-mutated proteins. Nonlimiting examples of such variants or mutants include factor IX K5R, factor IX K5A, factor IX V10K (wherein, e.g., the amino acid K is substituted for the amino acid Rat position 5 in the factor IX amino acid sequence), factor IX with substitutions at critical arginine amino acid sites in the catalytic domain of the protein including arginine 338 and arginine 333 (e.g., FIX R338A, FIX R338Q), and any combination thereof.

It is also further contemplated in this invention that an active fragment of a clotting factor protein can be administered to a subject of this invention. An active fragment comprises less than all of the amino acids of the full protein and comprises a sufficient number of amino acids of the protein to have the requisite or desired activity or at least 5%, 10%, 20%, 30%, 40%, 50%, 60%, 70%, 80%, 90%, 95% or 98% of the requisite or desired activity, which in the present invention, is to function in coagulation. A nonlimiting example of an active fragment of a coagulation protein of this invention is a factor VIII protein from which domain B has been removed (i.e., a factor VIII protein comprising, consisting essentially of and/or consisting of the five remaining factor VIII domains).

A fragment of a polypeptide or protein of this invention can be produced by methods well known and routine in the art. Fragments of this invention can be produced, for example, by enzymatic or other cleavage of naturally occurring peptides or polypeptides or by synthetic protocols that are well known. Such fragments can be tested for one or more of the biological activities of this invention (e.g., function in coagulation, function in reducing inflammation, function in supporting or opposing new blood vessel formation (angiogenesis), function in vitamin K binding)) according to the methods described herein, which are routine methods for testing activities of polypeptides, and/or according to any art-known and routine methods for identifying such activities. Such production and testing to identify biologically active fragments and/or immunogenic fragments of the polypeptides described herein would be well within the scope of one of ordinary skill in the art and would be routine.

The invention further provides homologues, as well as methods of obtaining homologues, of the polypeptides and/or fragments of this invention from other organisms included in this invention. As used herein, an amino acid sequence or protein is defined as a homologue of a polypeptide or fragment of the present invention if it shares significant homology to one of the polypeptides and/or fragments of the present invention. Significant homology means at least 65%, 75%, 80%, 85%, 90%, 95%, 98% and/or 100% homology with another amino acid sequence. Specifically, by using the nucleic acids that encode the clotting factor proteins and fragments of this invention (as are known in the art and incorporated by reference herein), as a probe or primer, and techniques such as PCR amplification and colony/plaque hybridization, one skilled in the art can identify homologues of the polypeptides and/or fragments of this invention in other organisms on the basis of information available in the art.

A subject of this invention is any subject that is susceptible to joint damage following bleeding into a joint space and/or susceptible to and/or having a clotting factor disorder. Nonlimiting examples of a subject of this invention include mammals, such as humans, nonhuman primates, domesticated mammals (e.g., dogs, cats, rabbits), livestock and agricultural mammals (e.g., horses, cows, pigs).

A joint of this invention in a human subject can include, but is not limited to, knee, ankle, wrist, finger, toe, hip, shoulder, elbow and any combination thereof. A joint of this invention in an equine subject can include, but is not limited to, metacarpus, metatarsus, fetlock, coffin, pastern, stifle and any combination thereof.

A subject of this invention can be “in need of” the methods of the present invention, e.g., because the subject has, or is believed at risk for, a disorder including those described herein and/or is a subject that would benefit from the methods of this invention. For example, a subject in need of the methods of this invention can be, but is not limited to, a subject diagnosed with, having or suspected to have, or at risk of having or developing a clotting factor disorder bleeding into a. joint.

In the methods of this invention that describe the delivery of a clotting factor protein or active fragment thereof and/or a nucleic acid encoding a clotting factor protein or active fragment thereof, the protein or nucleic acid can be delivered, for example, into synovial (i.e., joint) fluid, synovial tissue, muscle tissue within or in immediate proximity to the joint, isolated blood vessels supplying a joint, cartilage, chondrocytes, synoviocytes [e.g., fibroblast-like synoviocytes (FLS)], mesenchymal stem cells, platelet precursors, muscle cells, fibroblasts, and any combination thereof.

In some embodiments, the nucleic acid of this invention, encoding a clotting factor protein or active fragment thereof is in a vector, which can be a viral vector. In certain embodiments, the viral vector is an adeno-associated viral (AAV) vector, which can be an AAV vector of any of the AAV serotypes described herein. The AAV vector can also be a chimeric AAV vector as described herein. Nonlimiting examples of various AAV serotypes that can be employed in the methods of this invention (e.g., AAV2, AAV5, AAV8) are described in the Examples section provided herein and are well known in the art.

The methods of this invention can further comprise delivering an effective amount of an anti-inflammatory agent, an effective amount of a cytokine, or a combination thereof to the joint of the subject to reduce or prevent bleeding-associated joint damage. Nonlimiting examples of an anti-inflammatory agent of this invention include steroids and nonsteroid anti-inflammatory agents as are well known in the art. Nonlimiting examples of a cytokine of this invention include anti-inflammatory cytokines such as IL-10, IL-4, IL-11, IL1 Ra, TGF-13, osteoprotegerin and any combination thereof. The anti-inflammatory agents and cytokines of this invention can be delivered to the subject as a protein or active fragment thereof and/or as a nucleic acid encoding the protein or active fragment thereof. The amino acid sequences and nucleic acid sequences of the anti-inflammatory agents and cytokines of this invention, as well as active fragments thereof are well known in the art and would be readily available to those skilled in the art. The clotting factor proteins, anti-inflammatory agents and cytokines, either as proteins or nucleic acids can be administered in any combination and in any order relative to one another and in any time frame relative to one another.

In further embodiments of this invention, it is contemplated that a nucleic acid of this invention can be delivered to a subject of this invention, wherein the nucleic acid encodes a clotting factor protein or active fragment thereof, and/or an antagonist of a pro-inflammatory agent and the nucleic acid is under the control of a promoter and/or other regulatory element such that expression of the nucleic acid is induced by a pro-inflammatory agent to produce the clotting factor and/or antagonist of the pro-inflammatory agent. Nonlimiting examples of antagonists of pro-inflammatory agents include antagonists of TNFα, CSF-1, IL-6, IL 12, IL17, IL1B, receptor activator of nuclear factor-kappa B (RANK), RANK ligand (RANKL) and combinations thereof.

Embodiments are also provided herein wherein the methods of this invention can further comprise systemically delivering a coagulation or clotting factor protein or active fragment thereof to the subject by means of delivery directly to the joint and/or joint space. Also provided are methods of this invention that further comprise systemically delivering a nucleic acid encoding a coagulation or clotting factor protein or active fragment to the subject by means of delivery directly to the joint and/or joint space.

In yet additional embodiments of this invention, a method is provided of treating a clotting factor disorder in a subject that has antibodies that inhibit the activity of a clotting factor protein (e.g., antibodies to factor VIII and/or factor IX), comprising delivering an effective amount of a clotting factor protein or active fragment thereof to a joint space of the subject, thereby treating the clotting factor disorder in the subject.

Also provided herein is a method of treating a clotting factor disorder in a subject that has antibodies that inhibit the activity of a clotting factor protein (e.g., antibodies to factor VIII and/or factor IX), comprising delivering an effective amount of a nucleic acid encoding a clotting factor protein or active fragment thereof to the subject, thereby treating the clotting factor disorder in the subject.

Further provided herein is a method of treating a clotting factor disorder in a subject that has antibodies that inhibit the activity of a clotting factor protein (e.g., antibodies to factor VIII and/or factor IX), comprising delivering an effective amount of a clotting factor protein or active fragment thereof to a joint space of the subject and delivering an effective amount of a nucleic acid encoding a clotting factor protein or active fragment thereof to the subject, thereby treating the clotting factor disorder in the subject. Nonlimiting examples of clotting factors that can be delivered to a subject that has an antibody that inhibits the activity of a clotting factor protein include factor VII, factor VII (activated), prothrombin, thrombin, factor VIII, factor IX, factor IX (activated), factor X, factor X (activated), Tissue Factor, and any combination thereof.

Other embodiments of this invention include a method of maintaining and/or improving local hemostasis and/or reducing bleeding-associated bone and joint damage in a subject during and/or following a surgical procedure, comprising delivering an effective amount of a clotting factor protein or active fragment thereof to a joint space of the subject and/or delivering an effective amount of a nucleic acid encoding a clotting factor protein or active fragment thereof to the subject during and/or following a surgical procedure, thereby maintaining and/or improving local hemostasis and/or reducing bleeding associated bone and joint damage in the subject. Nonlimiting examples of a surgical procedure of this invention include joint replacement surgery; joint tissue repair; joint aspiration; concomitant administration of intra-articular radiation, sclerosing agents and/or corticosteroids; excision of a hemophilic pseudotumor and any combination thereof. A clotting factor protein of this invention can also be delivered within a hemophilic pseudotumor.

Further embodiments of this invention include a method of reducing or preventing post-operative bleeding and/or bleeding associated damage in a clotting factor deficient subject, comprising delivering an implantable matrix comprising an effective amount of a clotting factor protein or active fragment thereof to a joint space of the subject and/or delivering an effective amount of a nucleic acid encoding a clotting factor protein or active fragment thereof to the subject during and/or following a surgical procedure (e.g., joint replacement or any other surgical procedure in a clotting factor deficient subject that could produce post-operative bleeding and/or bleeding associated damage in the subject).

In some embodiments, the implantable matrix can comprise, consist essentially of and/or consist of an implantable device, a surgical graft material, a positively-charged nylon membrane, a suture, cat gut, a tissue scaffold, or a bone graft substitute. In certain embodiments, the implantable matrix can comprise, consist essentially of and/or consist of polytetrafluoroethylene (GORTEX™), poliglecaprone (MONOCRYL™), high density polyethylene (MARLEX™), polypropylene, polyglactin, polydiaxanone (PDS), or polyethylene terephthalate (DACRON™), as described in U.S. Pat. No. 7,201,898, the entire contents of which are incorporated by reference herein.

In addition, the present invention provides a method of maintaining and/or increasing hemostatic potential and/or reducing or preventing bleeding-associated joint and/or tissue damage within the joint space of a hemostatically normal subject (i.e., a subject that does not have or has not been diagnosed with an underlying bleeding disorder), comprising delivering an effective amount of a nucleic acid encoding a clotting factor protein or active fragment thereof to the subject, thereby maintaining and/or increasing hemostatic potential and/or reducing or preventing bleeding-associated joint and/or tissue damage within the joint space of the subject.

Also provided herein is a method of maintaining and/or increasing hemostatic potential and/or reducing or preventing bleeding-associated joint and/or tissue damage within the joint space of a hemostatically normal subject, comprising delivering an effective amount of a nucleic acid encoding a clotting factor protein or active fragment thereof to the subject, thereby maintaining and/or increasing hemostatic potential and/or reducing or preventing bleeding-associated joint and/or tissue damage within the joint space of the subject.

Further provided herein is a method of maintaining and/or increasing hemostatic potential and/or reducing or preventing bleeding-associated joint and/or tissue damage within the joint space of a hemostatically normal subject, comprising delivering an effective amount of a clotting factor protein or active fragment thereof to a joint space of the subject and delivering an effective amount of a nucleic acid encoding a clotting factor protein or active fragment thereof to the subject, thereby maintaining and/or increasing hemostatic potential and/or reducing or preventing bleeding-associated joint and/or tissue damage within the joint space of the subject.

Such a hemostatically normal subject can be, for example, a subject who does not have an underlying bleeding disorder but who may be at risk for bleeding-associated joint and/or tissue damage resulting from intra-articular bleeding as a result of trauma, such as an athlete (e.g., human, race horse, race dog), a recreational or amateur sports player and/or a subject having an occupation and/or hobby that places the subject at increased risk for trauma or damage to a joint.

The present invention also provides various compositions. In some embodiments these compositions can be employed, e.g., in the methods described herein. Thus, the present invention provides a composition comprising, consisting essentially of and/or consisting of a coagulation or clotting factor protein and/or active fragment thereof and a nucleic acid encoding a coagulation protein clotting factor protein and/or active fragment thereof, which can be, for example, in a pharmaceutically acceptable carrier. Such compositions of this invention can further comprise, consist essentially of and/or consist of an anti-inflammatory agent, a cytokine, an immune modulator, or any combination thereof and/or a nucleic acid encoding an anti-inflammatory agent, a cytokine, an immune modulator, or any combination thereof.

Additionally provided herein is a composition comprising, consisting essentially of and/or consisting of a coagulation protein or clotting factor protein and/or active fragment thereof and an anti-inflammatory agent, a cytokine, an immune modulator, or any combination thereof, which can be, for example, in a pharmaceutically acceptable carrier.

Further provided herein is a composition comprising, consisting essentially of and/or consisting of a nucleic acid encoding a clotting factor protein and/or active fragment thereof and a nucleic acid encoding an anti-inflammatory agent, a cytokine, an immune modulator, or any combination thereof, which can be, for example, in a pharmaceutically acceptable carrier.

It is further contemplated that the present invention provides a kit comprising, consisting essentially of and/or consisting of compositions of this invention. It would be well understood by one of ordinary skill in the art that the kit of this invention can comprise one or more containers and/or receptacles to hold the reagents (e.g., clotting factor proteins or active fragments thereof, nucleic acids, viral vectors) of the kit, along with appropriate buffers and/or diluents and/or other solutions and directions for using the kit, as would be well known in the art. Such kits can further comprise anti-inflammatory agents, antagonists of pro-inflammatory agents and/or other cytokines, in any combination, as described herein and as are well known in the art.

The compositions and kits of the present invention can also include other medicinal agents, pharmaceutical agents, carriers and diluents, etc. Actual methods of preparing such dosage forms are known, or will be apparent, to those skilled in this art.

In the kits of this invention, the compositions can be presented in unit\dose or multi-dose containers, for example, in sealed ampoules and vials, and can be stored in a freeze-dried (lyophilized) condition requiring only the addition of the sterile liquid carrier, for example, saline or water-for-injection immediately prior to use.

### EXAMPLES

### Example I

### Intra-Articular Factor IX Protein Replacement Protects Against Development of Hemophilic Synovitis in the Absence of Circulating Factor IX

Animal care and study. Wild type C57BL/6 mice were purchased from the Jackson Laboratory (Bar Harbor, Me.). Factor IX knockout (FIX−/−) mice^{1,3} were bred in house. All investigations were approved by the UNC-CH Institutional Animal Care and Use Committee. Mice were anesthetized using intraperitoneal 1.25% Avertin for all procedures. Knee joint intra-articular bleeding challenge was performed using a Hamilton syringe with 30.5G needle via a small (˜0.5 mm) incision of the skin overlying the patella as described.¹² All blood samples were collected from the retro-orbital plexus into 1:9 parts 3.2% citrated sodium and stored at −80° C. The knee joints were collected by sectioning the femur and tibia 1-cm from the joint, fixed, and decalcified using routine histological procedures. ¹. Jin D Y, Zhang T P, Gui T, Stafford D W, Monahan P E. Creation of a mouse expressing defective human factor IX. Blood. 2004;104:1733-1739.³. Lin H F, Maeda N, Smithies O, Straight D L, Stafford D W. A coagulation factor IX-deficient mouse model for human hemophilia B. Blood. 1997;90:3962-3966.¹². Narine Hakobyan C E, Ada A Cole, D. Rick Sumner and Leonard A. Valentino. Experimental Haemophilic arthropathy in a mouse model of a massive hemarthrosis: Gross, radiological, and histological changes. Haemophilia. 2008, In press.

Histologic grading. Hemophilic synovitis in injured and uninjured joints was graded according to a validated system. At least three representative fields from an equatorial section of each knee were scored by three or more reviewers who were blinded to the experimental conditions. The total synovitis scores from each joint were averaged. Images were captured with a DMX-1200 color camera using the Act1 software (Nikon).^{4,5} ⁴. Morko J, Kiviranta R, Joronen K, Saamanen A M, Vuorio E, Salminen-Mankonen H. Spontaneous development of synovitis and cartilage degeneration in transgenic mice overexpressing cathepsin K. Arthritis Rheum. 2005;52:3713-3717.⁵. Valentino L A, Hakobyan N, Kazarian T, Jabbar K J, Jabbar A A. Experimental haemophilic synovitis: rationale and development of a murine model of human factor VIII deficiency. Haemophilia. 2004;10:280-287.

Development of hemophilic synovitis model in mice. Bleeding into the joints of hemophilic mice leads to clinical and pathological changes that closely resemble the hemophilic synovitis that develops in hemophilia patients. The use of a blunt injury bleeding model in hemophilia A mice has been described.⁵ A more robust model of blood induced joint damage (BIJD) was developed, consisting of a single 30.5 gauge needle puncture of the knee joint capsule to induce hemarthrosis. This model was adapted for use in FIX^{-/-}mice. The specificity of the synovitis induced by capsular puncture injury was validated in large cohorts of wild type mice (n=28) and FIX^{-/-}mice (n=58). The single needle puncture does not result in arthropathy in hemostatically normal mice; the mean synovitis grade was 0.14±0.3 and no mice developed synovitis graded at ≧2 (scale of increasing pathology from 0-10). In contrast, the same injury results in synovitis (histopathology grade of ≧2) in >96% control hemophilia B mouse joints, with a mean synovitis grade of 4.4±2.

In vivo administration of intra-articular and systemic coagulation protein. Study groups of FIX−/−mice received recombinant human factor IX (rhFIX, BeneFix®, Genetics Institute, Inc, Cambridge, Mass., USA) at a range of doses injected either IV via tail vein or intra-articularly (IA) via a 30.5 gauge needle inserted into the left hind limb knee joint. Mice receiving IV factor IX then received a single puncture of the left hind knee joint capsule with a 30.5 gauge needle within fifteen minutes of the IV dose and intra-articular instillation of 5 μl normal saline (NS), to reproduce the bleeding challenge experienced by the IA study groups. Fourteen days after injury, both knee joints were collected for histological examination (≧3 animals in each treatment group). rhFIX doses for IA study groups were: 20 IU/kg (≈0.5 IU); 10 IU/kg (≈0.25 IU); 5 IU/kg(≈0.125 IU); or 2.5 IU/kg(≈0.0625 IU). Total volume for all doses was 5 μl. rhFIX doses for IV study groups were: 100 IU/kg; 50 IU/kg; and 25 IU/kg. A negative control group received the joint puncture injury with 5 μl saline IA, without any rhFIX.

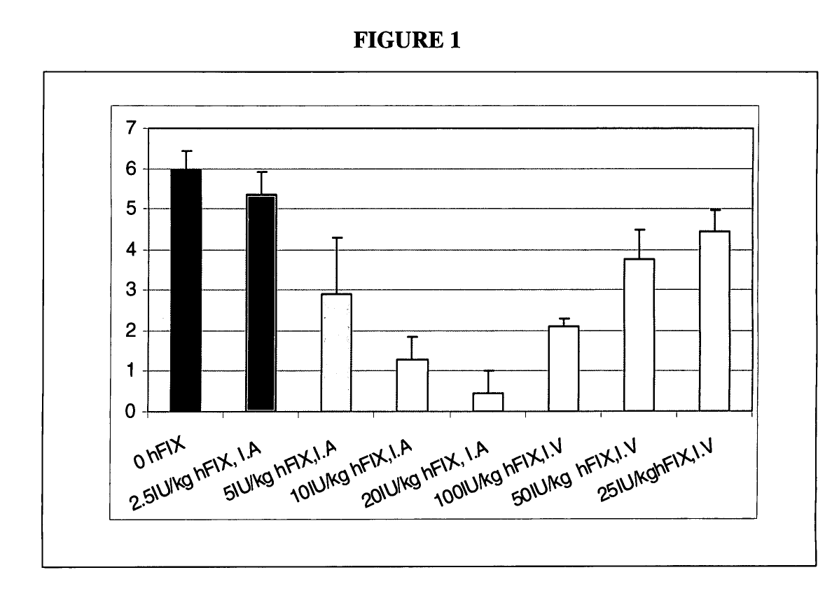

Intra-articular delivery of FIX protein provides protection from bleeding-induced synovitis To investigate the possibility that FIX present in the joint space can protect from BIJD in the absence of circulating FIX, a range of doses of recombinant hFIX was given either IA or IV coincident with the capsular puncture injury. As shown in FIG. 1, IA hFIX afforded significant protection against blood-induced synovitis. Pathological scores in mice treated with 5 IU/kg IA were equivalent or superior to those of mice treated with 50 IU/kg IV human factor IX. IA treatment at the highest dose (20 IU/kg) resulted in minimal synovitis (mean score 0.3).

Histopathological analysis revealed that in the wild type (hemostatically normal) mouse knee joint after needle puncture, the joint space is well maintained with a normal 3-4 cell layer synovial lining and no synovial hypervascularity. In the FIX−/−mouse joint after needle puncture and IA NS injection, gross blood is seen in the joint space, which is narrowed by synovial proliferation. In the FIX−/−mouse joint after IV recombinant hFIX 50 IU/kg followed by needle puncture and IA NS treatment, hypervascularity and synovial thickening (>8-10 cell layers) are present. In the FIX−/−mouse joint after needle puncture with coincident IA injection of hFIX 25 IU/kg, the joint space is well maintained with thin synovial lining and smooth cartilage.

FIX functional activity and anti-hFIX Bethesda inhibitor assay. One-stage factor IX activity assay (FIX-specific aPTT) and factor IX Bethesda inhibitor antibody assay were performed as previously described, using a START 4 Coagulation Analyzer (Diagnostica Stago, Asnières, France).⁶ ⁶. Zhang T P, Jin D Y, Wardrop R M, 3rd, et al. Transgene expression levels and kinetics determine risk of humoral immune response modeled in factor IX knockout and missense mutant mice. Gene Ther. 2007;14:429-440.

Circulating FIX after intra-articular versus intravenous delivery. A single dose of 20 IU/kg IA or 20 IU/kg or 80 IU/kg IV was given and citrated plasma was collected at 15 min, 1 hr and 2 hr after injection to examine FIX recovery. To examine extended survival of FIX, both 25 IU/kg or 100 IU/kg hFIX doses were studied IA and IV Citrated plasma collected at 1 hr, 4 hr, 12 hr, 24 hr, 48 hr, and 72 hr was studied in a one-stage factor IX activity assay.

Intra-articular FIX does not increase circulating FIX activity. The possibility was addressed next that, through technical error or other mechanism, the IA hFIX in fact enters the circulation and effects joint protection via the systemic plasma activity. The circulating pharmacokinetics of plasma FIX activity was examined following hFIX dosing via the tail vein or IA. It was determined that mouse

FIX and human FIX are not interchangeable in plasma. An IV dose of hFIX concentrate that would be expected to fully correct a deficient human (80-100 IU/kg of body weight) only partially corrected the FIX−/−mouse to 10-15% activity and this was the highest dose of hFIX used in any of the treatment groups shown in FIG. 1 and in Table 1. Although IA treatment at doses of 20 IU/kg or lower protected against BIJD (FIG. 1), IA treatment at this dose did not result in any detectable circulating FIX activity in the first two hours after treatment (Table 1).

Previous studies have shown that FIX can be given subcutaneously, intramuscularly, or intratracheally and via each of these routes, FIX will enter the circulation in a delayed fashion when compared to intravenous delivery.^{9,10} Therefore, an extended pharmacokinetic study was performed to rule out the possibility that FIX might enter the circulation from a local “depot” after IA injection. In a control group of mice treated IV, FIX activity decayed with a half-life of 7-12 hours, consistent with previous experience in this lab and others.¹¹ Even using a four-fold higher IA hFIX dose than was used in the joint protection studies (FIG. 1), no FIX activity was detected in plasma up to 72 hours after IA delivery (80 Ili/kg IA; Table 2). ⁹. Liles D, Landen C N, Monroe D M, et al. Extravascular administration of factor IX: potential for replacement therapy of canine and human hemophilia B. Thromb Haemost. 1997;77:944-948.¹⁰. Russell K E, Olsen E H, Raymer R A, et al. Reduced bleeding events with subcutaneous administration of recombinant human factor IX in immune-tolerant hemophilia B dogs. Blood. 2003;102:4393-4398.¹¹. Gui T, Lin H F, Jin D Y, et al. Circulating and binding characteristics of wild-type factor IX and certain Gla domain mutants in vivo. Blood. 2002;100:153-158.

Statistical analysis. Quantitative data are presented as means plus or minus SD. The two-tailed paired Student's t test was performed with a statistical software package (SAS v9.3). P values of less than 0.05 were considered a statistically significant difference.

In summary, the current standard of care for hemophilic arthropathy is to replace circulating plasma factor activity intravenously in either a reactive or prophylactic fashion and the data presented in these studies suggest that directing factor replacement to the hemophilic joint could yield therapeutic benefits that augment standard systemic therapy. To perform these studies in FIX^{-/-}mice required that a mouse bleeding model be established in which a single joint injury reliably reproduces in the hemophilia B animal the same pathology seen in human hemarthropathy, but does not result in joint pathology in hemostatically normal animals. This model shows for the first time that clotting factor IX within the joint space protects from the development of synovitis, in the absence of measurable circulating FIX activity or protein.

### Example II

### Extravascular Clotting Factor Activity within Joint Tissues Protects Against Progression of Hemophilic Arthropathy

The present study was carried out to determine whether replacement of deficient factor VIII within an injured joint capsule of mice with hemophilia A (FVIII−/−) would decrease the progression of synovitis.

A bleeding mouse model (described in Example I) was used, consisting of a unilateral knee joint capsule needle puncture to induce hemorrhage in hemophilic mice. Pathology of the joint at two weeks after the injury was graded 0 to 10 using a murine hemophilic synovitis grading system.

Coincident with needle puncture, recombinant human coagulation factor doses ranging from 0 to 25 IU/kg of factor VIII were instilled intraarticularly (IA). Comparison groups received the same injury and intravenous (IV) factor VIII doses of 25 IU/kg to 100 Ili/kg (n=4-7 mice per study group).

Joint bleeding phenotype of the two strains of mice was similar. Mice receiving only saline injection at the time of needle puncture developed mean synovitis scores of 5±0.5 in the FVIII−/−mice. Protection by human clotting factor in the mouse coagulation system was incomplete; mice receiving 100 IU/kg I.V. of factor VIII developed synovitis scores of 2.6±1.7. In contrast, the pathology grade of FVIII−/−mice dosed with 25 IU/kg IA was 0.67±0.3 (p=0.05 for comparison of 25

IU/kg IA with 100 IU/kg IV).

Additional experiments were done to rule out the possibility that clotting factor that was delivered IA was entering the circulation and resulting in joint protection via that route, either through technical error at the time of injection, or from a depot effect in the joint with late equilibration into the circulation.

Additional groups of mice received factor VIII intravenously at 100 IU/kg, or intraarticularly at four times the doses used in the hemarthrosis challenge (100 IU/kg FVIII) and factor activity assays were performed at 1, 4, 12, 24, and 48 hours. Expected circulation kinetics were seen following IV dosing; no increase in circulating factor VIII activity was seen in the intraarticular dosing groups at any time point.

In considering the potential immunogenicity of an intraarticular therapy approach for hemophilic joint therapy, factor VIII−/−mice were treated with three doses of human factor VIII, 100 IU/kg, at five day intervals either IV or IA At two weeks after exposure, 5/5 IV-treated mice developed inhibitor antibodies with titers in the range of 0.8-7.2 BU; 2/5 IA-treated mice had detectable low-titer antibodies (1.3 BU), indicating no greater immunogenicity in the IA model. Extravascular factor VIII can contribute to protection against blood-induced joint deterioration and enhancing local tissue hemostasis with FVIII protein and/or gene therapy could prove to be a useful adjunct to systemic replacement.

### Example III

### Intra-Articular Coagulation Factor IX Gene Replacement Protects Against Development of Hemophilic Synovitis in the Presence or the Absence of Circulating Factor IX

AAV vector constructs and production. The AAV vector containing the hFIX cDNA (1.4 kb) under transcriptional control of chicken β-actin (CBA) promoter (rAAV-CBA-hFIX) has been described previously.¹ All vectors were produced and titered at the UNC Virus Vector Core Facility as described previously.² ². Xiao X, Li J, Samulski R J. Efficient long-term gene transfer into muscle tissue of immunocompetent mice by adeno-associated virus vector. J Virol. 1996;70:8098-8108.

Synovial cell culture and ex vivo tissue explant transduction by AAV-GFP. All cell lines and tissue explants were provided by the Musculoskeletal Tissue Bank of the UNC-CH Thurston Arthritis Research Center and were collected with informed patient consent following approval by the UNC Internal Review Board for Human Studies. Primary cells lines of fibroblast-like synoviocytes (FLS) from a healthy individual and one with osteoarthritis were transduced by self complementary AAV (scAAV) vectors encoding green fluorescent protein (GFP) at a multiplicity of infection (MOI) of 5,000 vg/cell. Serotypes used were AAV2, 5 and 8. In addition, fresh tissue explants were retrieved from the operating room; cartilage (100 mg fragments) or synovium (20 mg fragments) was plated in tissue culture medium and were overlaid with 5×10⁹ vg (in 50 μl) GFP-expressing vectors. Four days later, tissues were fixed in 4% PFA overnight, frozen in OCT, and sectioned at 5 μm for fluorescence examination.

AAV transduction of chondrocytes and synoviocytes in human joint tissue explants and cultured cell lines. The potential for AAV gene therapy vectors to transduce individual cell types within the synovium and the cartilage after gene delivery to the synovial space was analyzed. Monolayer tissue cultures of primary fibroblast-like synoviocytes (FLS) cell lines were transduced at an MOI of 5000 vg/cell using scAAV vectors expressing the Green Fluorescent Protein (GFP) and packaged in AAV capsids of serotypes 2, 5, or 8. Five days later, serial sections were observed for fluorescence. Cartilage from fresh joint tissue explants maintained in tissue culture were overlaid with 5×10⁹ scAAV GFP packaged in serotypes 2, 5 or 8. Four days later, serial sections were observed for fluorescence.

The fluorescence images demonstrated that the AAV2 vector directed the strongest GFP expression in FLS, followed by the AAV5 vector, with virtually no transduction from AAV8 in vitro. Chondrocyte biology is altered when cells are not studied within the rich extracellular matrix of cartilage that they produce, so cartilage transduction was studied by overlaying AAV-GFP virus on fresh cartilage surgical explants. While most vectors transduced some chondrocytes, AAV8 transduction was most efficient, followed by AAV5 and AAV2. The contrasting tropisms of AAV serotypes, even within cell subpopulations of a single tissue, were demonstrated by comparing AAV2 (strong GFP gene expression in FLS/weak expression in chondrocytes) with AAV8 (minimal expression in FLS/strong transduction in chondrocytes).

Bioluminescence imaging of mice. Under isoflurane anesthesia, mice were injected intraperitoneally with 150 μg/g D-luciferin in PBS. Bioluminescence imaging with a CCD camera (IVIS, Xenogen) was initiated exactly 15 min after injection. Signal intensities from regions of interest are expressed as total photon flux (photons/s/cm²).⁸ ⁸. Bloquel C, Trollet C, Pradines E, Seguin J, Scherman D, Bureau M F. Optical imaging of luminescence for in vivo quantification of gene electrotransfer in mouse muscle and knee. BMC Biotechnol. 2006;6:16.

Differential gene expression from AAV serotypes examined using serial in vivo bioluminescence imaging. Having established that joint tissues could be efficiently transduced in vitro, the ability to localize in vivo expression using IA injection was studied using AAV vectors expressing firefly luciferase, a marker gene the expression of which could be followed serially in joints. Single strand AAV.Iuciferase vectors encapsidated in serotypes AAV2, AAV5 or AAV8 were injected into the left knee joint of adult mice at a dose of 8×10⁸ vg/animal. Bioluminescence imaging with a CCD camera (IVIS, Xenogen) was initiated and recorded exactly 15 minutes after injection with D-luciferin, the substrate of luciferase. After acquiring a gray-scale photograph, a bioluminescent image was captured with adjusted exposure time, binning (resolution) factor, 1/f stop and open filter to acquire maximum signal while avoiding a saturated image. Intensity and biodistribution of luciferase expression were imaged weekly.

AAV2, AAV5 and AAV8 luciferase vectors all transduced joints in vivo. Expression was primarily confined to the injected joint 1 week after infection, although some luciferase expression from the AAV8 vector could be seen in extraarticular sites at that time point. By four weeks post-infection, the majority of signal from the AAV8 vector came from the hepatosplenic region, at a time the other serotypes localized expression to the articular or periarticular space.

In vivo administration of intra-articular coagulation protein gene therapy. At the time of delivery of gene therapy IA, FIX−/−mice were given hemostatic protection using 100 IU/kg hFIX IV, then were injected in the left knee with either lower dose (2.5×109 vector genomes (vg)) or higher dose (1.0×1010 vg) AAV2, AAV5 and AAV8 vectors in a total volume of 5 μl. The right knee received a capsular puncture as well and 5 μl NS injection as control. After 4 weeks, bilateral knee injury was induced by needle puncture. Two weeks later, joints were harvested and pathology was graded.

Immunohistochemistry of hFIX. Paraffin embedded samples were sectioned (5 μm). Following deparafinization, rehydration, and blocking with 3% BSA for 30 min, polyclonal rabbit anti-hFIX (DAKO, Carpinteria, Calif., USA) diluted 1:200 in PBS/1% bovine serum albumin was added and incubated overnight at 4° C. After rinses with PBS-T, a HRP conjugated secondary antibody was applied for 20 minutes at 25° C. Slides were washed and incubated with DAB, and counterstained with haematoxylin.⁷ The total number of cells and number of HRP-positive cells in six representative fields were enumerated and the percentage of positive cells calculated. ⁷. Wu Z, Sun J, Zhang T, et al. Optimization of Self-complementary AAV Vectors for Liver-directed Expression Results in Sustained Correction of Hemophilia B at Low Vector Dose. Mol Ther. 2007.

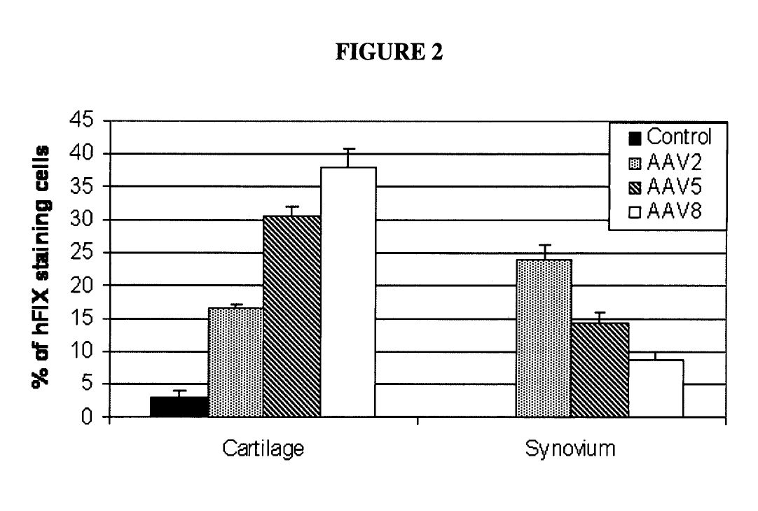

Intra-articular delivery of AAV.hFIX: Biodistribution of expression within and outside the joint. Prior to subjecting hemophilic animals to an injury, studies were conducted to document, using the therapeutic gene of interest, relative biodistribution of expression from the AAV serotype vectors. AAV2-, AAV5- and AAV8-CBA-hFIX vectors were injected into the knee joints of FIX^{-/-}mice at the dose of 2.5×10⁹ vg/animal. Four weeks later, knee joints were harvested and immunohistochemical staining for human factor IX was performed. The anti-hFIX antibody was shown not to cross-react with mouse FIX in untreated wild type mouse joint. As shown in FIG. 2, patterns of serotype tropism were consistent with those seen in vitro: AAV5 transduced both FLS and chondrocytes, while AAV2 demonstrated a bias toward FLS transduction and AAV8 toward chondrocyte transduction. In addition, knee joint and liver tissues of treated animals were homogenized and assayed by hFIX ELISA. Minimal or no FIX was detected in liver when compared to joint tissue treated with AAV 2 or AAV5, while the majority of FIX was intrahepatic after AAV8 delivered IA.

FIX functional activity and anti-hFIX Bethesda inhibitor assay. One-stage factor IX activity assay (FIX-specific aPTT) and factor IX Bethesda inhibitor antibody assay were performed as previously described, using a START 4 Coagulation Analyzer (Diagnostica Stago, Asnieres, France).⁶

Intra-articular AAV-hFIX protects against development of hemophilic synovitis. Examined next was the potential for IA AAV-directed expression of hFIX to protect the joint from subsequent blood-induced injury (capsular puncture). Under hemostatic coverage at the dose of 100 IU/kg body weight with IV rhFIX, FIX^{-/-}mice (4 mice/group) were injected in the left hindlimb joint with either a lower dose (2.5×10⁹ vg) or a higher dose (1×10¹⁰ vg) of ssAAV2-, ssAAV5-, or ssAAV8-CBA-hFIX vectors. These doses are equivalent to approximately 1×10¹¹ vg/kg body weight (lower dose) and 4×10¹¹ vg/kg (higher dose). Normal saline was injected into the right hindlimb joint under hemostatic coverage with IV FIX. After 4 weeks of vector expression, both knees were subjected to capsular puncture to induce hemarthrosis. Two weeks later, joints were harvested and histopathologic synovitis grading was performed, comparing the animal's treated hindlimb to injured untreated control hindlimb.

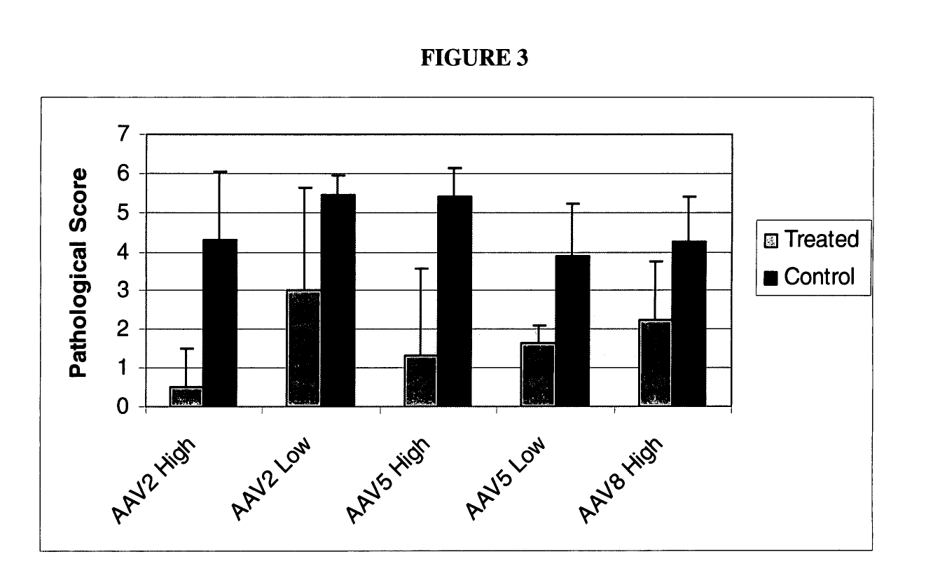

The injured untreated knee showed changes of hemarthropathy, including synovial proliferation narrowing the joint space, synovial thickening, foci of hypervascularity and synovial overgrowth of the tibial articular surface. Following gene therapy, most mice in the higher dose AAV2- or AAV5-treated groups showed minimal synovitis and most joints scored 0-1 (P<0.01 for the difference between injured contralateral control limbs and both ssAAV2.hFIX and ssAAV5.hFIX groups) (FIG. 3). There was considerable inter-animal variability between the animals in most of the AAV-treated groups. ssAAV8-CBA-hFIX treatment at this dose also resulted in significant protection of treated joints (treated versus control knee, P=0.03), although none of the mice treated with AAV8 showed complete protection (mean score 2.22). The four-fold lower dose of ssAAV2-CBA-hFIX and ssAAV8-CBA-hFIX did not demonstrate significant protection. ssAAV5-CBA-hFIX at the lower dose was also protective (P=0.03).

The AAV2 and AAV5 treated animals did not have detectable circulating FIX at sacrifice (6 weeks post-vector treatment) to account for the protective effect within the joint, as measured by ELISA or one-stage FIX activity assay. Low levels of FIX circulated in some AAV8-treated mice (10-300 ng/ml FIX antigen and <1%-7.3% activity), consistent with the hepatic spread of AAV8 after IA virus delivery as demonstrated by the luciferase imaging. None of the mice had anti-FIX antibodies detected by the Bethesda assay during the six weeks following ssAAV-CBA-hFIX dose. This finding was unexpected, because this strain of FIX^{-/-}mice reliably develops neutralizing anti-human factor IX antibodies following intramuscular treatment with ssAAV2-CBA-hFIX vector^{1,6}.

Statistical analysis Quantitative data are presented as means plus or minus SD. The two-tailed paired Student's t test was performed with a statistical software package (SAS v9.3). P values of less than 0.05 were considered a statistically significant difference.

In summary, the current standard of care for hemophilic arthropathy is to replace circulating plasma factor activity intravenously in either a reactive or prophylactic fashion and the data presented in these studies suggest that directing factor replacement to the hemophilic joint could yield therapeutic benefits that augment standard systemic therapy. To perform these studies in FIX^{-/-}mice required that a mouse bleeding model be established in which a single joint injury reliably reproduces in the hemophilia B animal the same pathology seen in human hemarthropathy, but does not result in joint pathology in hemostatically normal animals. This model shows for the first time that clotting factor IX within the joint space protects from the development of synovitis, even in the absence of measurable circulating FIX activity or protein. These studies establish that persistent expression of FIX following AAV gene therapy in the joint may contribute to protection from bleeding induced joint pathology.

### REFERENCES FOR EXAMPLE III

### Example IV

### Point Mutations in the Factor IX Gla-Domain Yield Proteins with Altered Function in Coagulation

The amino terminal residues 3-11 of the factor IX Gla domain have been shown to be responsible for binding to endothelial cells. The recruitment of circulating factor IX to the platelet surface is a critical step in thrombus formation and the platelet phospholipid surface is a critical component of the calcium-dependent factor X activation complex formed by factor IX with factor VIII. FIX has been shown to bind specifically to extracellular matrix collagen IV via the Gla-moieties. Point mutations in this region were targeted, with the result that a FIX with a mutation of lysine to alanine at amino acid 5 (FIX K5A) or of valine to lysine at amino acid 10 (FIX V10K) results in loss of binding to endothelial cells. In contrast, a mutation of lysine to arginine at residue 5 results in a FIX (FIX K5R) molecule that has a 3-fold increased affinity for the endothelial binding site. When infused into mice at equimolar amounts, the K5R mutant rapidly distributed out of the circulation to the endothelium and the non-binding mutants showed longer circulating kinetics. This functional difference has been exploited to achieve higher circulating levels of factor IX after intramuscular gene therapy by using a K5A factor IX transgene that was not subject to the wild type factor IX transgene's local sequestration in extracellular sites in muscle.

To test for the in vivo role of factor IX binding to collagen IV, a mouse has been created in which endogenous factor IX has been replaced by mouse FIX K5A. The K5A mouse has been shown to have more circulating factor IX than the wild type mouse, although the amount of mRNA is less than that found in the wild type animal. This reflects the decreased binding of K5A factor IX to collagen IV. Interestingly there is no detectable difference between the K5A factor IX protein and wild type factor IX protein when tested in the one stage factor IX activated partial thromboplastin time assay. Also, there is no detectable difference between the thrombus generated in a K5A mouse and a wild type mouse in a laser induced thrombosis model (which is not expected to expose basement membrane collagen IV). However, when the injury is induced by ferric chloride, which exposes collagen (including collagen IV), the time for occlusive thrombus formation observed by intravital microscopy is significantly slower in the K5A mouse as opposed to wild type. Additionally, a tail bleeding assay, modified to include observations that quantify late rebleeding, demonstrates delayed but ultimately effective hemostasis in the K5A mouse when compared to wild type. These results suggest that the factor IX molecule with decreased affinity for collagen IV fails to support either hemostasis or thrombus generation at a normal rate even though platelet recruitment is normal. A K5R factor IX described by our group that has avid binding to the endothelium and increased affinity for collagen IV will potentially generate enhanced hemostasis and thrombosis in a local fashion. For specific use in reducing bleeding-induced joint damage, the K5R factor IX's property of binding to collagen IV should enable K5R factor IX to be sequestered locally in the joint. This and other factor IX proteins containing point mutations in the protein's Gla-domain can enhance hemostasis within the joint and/or direct a more prolonged bioavailability specifically in the joint space, which has a rich supply of collagen IV. Additional mutations (including mutations at critical arginine amino acids in the factor IX catalytic domain, e.g. FIX R338A, FIX R338Q) could be combined with the Gla-domain mutant factor IX which increase the specific activity of the protein that is localized to the joint, with minimal risk of causing thrombosis, which would be a safety concern of using high specific activity proteins intravenously in the general circulation.

The foregoing is illustrative of the present invention and is not to be construed as limiting thereof. Although a few exemplary embodiments of this invention have been described, those skilled in the art will readily appreciate that may modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein. All publications, patent applications, patents, patent publications, sequences (nucleotide sequences, single polymorphism nucleotides, amino acid sequences, etc.) identified in the GenBank® database or other sequence databases and any other references cited herein are incorporated by reference in their entireties for the teachings relevant to the sentence and/or paragraph in which the reference is presented.

TABLE 1
Initial circulating FIX activity in mouse plasma after recombinant hFIX

| Mouse | Dose/route | 15 min | 1 hr | 2 hr |
|---|---|---|---|---|
| FIX^{-/-} | 20 IU/kg IA | <1% | <1% | <1% |
| FIX^{-/-} | 20 IU/kg IV | 1.95 ± 0.57 | 1.63 ± 0.68 | <1% |
| FIX^{-/-} | 80 IU/kg IV | 15.7 ± 5.88 | 11.5 ± 5.56 | 8.75 ± 3.46 |

FIX^{-/-} mice were given recombinant hFIX by IV or IA at the dose of 20 IU/kg or 80 IU/kg. Citrated plasma was collected at 15 min, 1 hr and 2 hr for FIX activity assay (aPTT).
IA, intra-articular;
IV, intravenous by tail vein

TABLE 2
Extended circulating FIX activity in mouse plasma after recombinant
hFIX

| Mouse | Dose/route | 1 hr | 4 hr | 12 hr | 24 hr | 48 hr | 72 hr |
|---|---|---|---|---|---|---|---|
| FX^{-/-} | 25 IU/kg | <1% | <1% | <1% | <1% | <1% | <1% |
| (n = 6) | IA | | | | | | |
| FIX^{-/-} | 100 IU/kg | <1% | <1% | <1% | <1% | <1% | <1% |
| (n = 7) | IA | | | | | | |
| FIX^{-/-} | 25 IU/kg | 4.5 ± 0.9 | 2.1 ± 0.6 | 1.4 ± 0.4 | 1.0 ± 0.6 | <1% | <1% |
| (n = 8) | IV | | | | | | |
| FIX^{-/-} | 100 IU/kg | 17.3 ± 3.3 | 11.8 ± 4.8 | 6.4 ± 2.1 | 3.8 ± 1.8 | 2.7 ± 1.1 | 1.5 ± 0.4 |
| (n = 9) | IV | | | | | | |

FIX^{-/-} mice were given recombinant hFIX by IV or IA at the dose of 25 IU/kg or 100 IU/kg.
Citrated plasma was collected at 1 hr, 4 hr, 12 hr, 24 hr, 48 hr, and 72 hr for FIX activity assay (aPTT).
IA, intra-articular;
IV, intravenous by tail vein

TABLE 3

| | GenBank |
|---|---|
| | Accession |
| | Number |
| Complete | |
| Genomes | |
| Adeno- | NC_002077, AF063497 |
| associated | |
| virus 1 | |
| Adeno- | NC_001401 |
| associated | |
| virus 2 | |
| Adeno- | NC_001729 |
| associated | |
| virus 3 | |
| Adeno- | NC_001863 |
| associated | |
| virus | |
| 3B | |
| Adeno- | NC_001829 |
| associated | |
| virus 4 | |
| Adeno- | Y18065, AF085716 |
| associated | |
| virus 5 | |
| Adeno- | NC_001862 |
| associated | |
| virus 6 | |
| Avian | AY186198, |
| AAV | AY629583, |
| ATCC | NC_004828 |
| VR-865 | |
| Avian | NC_006263, |
| AAV | AY629583 |
| strain | |
| DA-1 | |
| Bovine | NC_005889, |
| AAV | AY388617 |
| Clade A | |
| AAV1 | NC_002077, AF063497 |
| AAV6 | NC_001862 |
| Hu.48 | AY530611 |
| Hu43 | AY530606 |
| Hu44 | AY530607 |
| Hu46 | AY530609 |
| Clade B | |
| Hu.19 | AY530584 |
| Hu.20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| HuT41 | AY695378 |
| HuS17 | AY695376 |
| HuT88 | AY695375 |
| HuT71 | AY695374 |
| HuT70 | AY695373 |
| HuT40 | AY695372 |
| HuT32 | AY695371 |
| HuT17 | AY695370 |
| HuLG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 | AY530579 |
| (AAV9) | |
| Hu31 | AY530596 |
| Hu32 | AY530597 |
| Clonal | |
| Isolate | |
| AAV5 | Y18065, |
| | AF085716 |
| AAV3 | NC_001729 |
| AAV3B | NC_001863 |
| AAV4 | NC_001829 |
| Rh34 | AY243001 |
| Rh33 | AY243002 |
| Rh32 | AY243003 |

1. A method of treating a blood clotting factor disorder in a subject, comprising delivering an effective amount of a clotting factor protein or an active fragment thereof to a joint space of the subject, thereby treating the blood clotting factor disorder in the subject.

2. A method of controlling bleeding and/or reducing bleeding-associated joint damage in a joint of a subject, comprising delivering an effective amount of a clotting factor protein or an active fragment thereof to a joint space of the subject, thereby controlling bleeding and/or protecting from further bleeding in the joint of the subject.

3. A method of treating a blood clotting factor disorder in a subject, comprising delivering an effective amount of a nucleic acid encoding a clotting factor protein or an active fragment thereof to a joint space of the subject, thereby treating the blood clotting factor disorder in the subject.

4. A method of controlling bleeding and/or reducing bleeding-associated joint damage in a joint of a subject, comprising delivering an effective amount of a nucleic acid encoding a clotting factor protein or an active fragment thereof to a joint space of the subject, thereby controlling bleeding in the joint space of the subject.

5. A method of treating or preventing hemophilic arthropathy or bleeding-associated joint damage in a subject, comprising delivering an effective amount of a clotting factor protein or an active fragment thereof to a joint space of the subject, thereby treating or reducing hemophilic arthropathy or bleeding-associated joint damage in the subject.

6. A method of treating or preventing hemophilic arthropathy or bleeding-associated joint damage in a subject, comprising delivering an effective amount of a nucleic acid encoding a clotting factor protein or an active fragment thereof to a joint space of the subject, thereby treating or reducing hemophilic arthropathy or bleeding-associated joint damage in the subject.

7. The method of claim 1, further comprising delivering an effective amount of a nucleic acid encoding a clotting factor protein or an active fragment thereof to a joint space of the subject.

8. The method of claim 1, further comprising systemically delivering an effective amount of a clotting factor protein or an active fragment thereof to the subject.

9. The method of claim 1, further comprising systemically delivering an effective amount of a nucleic acid encoding a clotting factor protein or an active fragment thereof to the subject.

10. The method of claim 1, wherein the clotting factor protein is selected from the group consisting of Factor VII, Factor VIIA (activated), Factor VIII, Factor IX, Factor X, Factor XI, von Willebrand factor, Protein C, activated Protein C, Protein S, bone Gla protein (osteocalcin), matrix Gla protein, prothrombin and any combination thereof.

11. The method of claim 3, wherein the nucleic acid encoding the clotting factor protein or active fragment thereof is in a viral vector.

12. The method of claim 11, wherein the viral vector is an adeno-associated viral vector.

13. The method of claim 1, further comprising delivering an effective amount of an anti-inflammatory agent, a cytokine, an immune modulator or any combination thereof to the subject.

14. The method of claim 1, further comprising delivering an effective amount of a nucleic acid encoding an anti-inflammatory agent, a cytokine, an immune modulator or any combination thereof to the subject.

15. The method of claim 1, wherein the subject is a mammal.

16. The method of claim 15, wherein the mammal is a human.

17. A composition comprising a clotting factor or active fragment thereof and a nucleic acid encoding a clotting factor protein or active fragment thereof, in a pharmaceutically acceptable carrier.

18. The composition of claim 17, further comprising an anti-inflammatory agent, a cytokine, an immune modulator, or any combination thereof.

19. The composition of claim 17, further comprising a nucleic acid encoding an anti-inflammatory agent, a cytokine, an immune modulator, or any combination thereof

20. A composition comprising a clotting factor protein or active fragment thereof and an anti-inflammatory agent, a cytokine, an immune modulator, or any combination thereof, in a pharmaceutically acceptable carrier.

21. A composition comprising a nucleic acid encoding a clotting factor protein or active fragment thereof and a nucleic acid encoding an anti-inflammatory agent, a cytokine, an immune modulator, or any combination thereof, in a pharmaceutically acceptable carrier.
